(19) United States
(12) Patent Application Publication
(10) Pub. No.: US 2015/0283226 A1
(43) Pub. Date: Oct. 8, 2015

(54) COMPOSITIONS AND METHODS OF MODULATING AN IMMUNE RESPONSE

(71) Applicants: Wilfred Jefferies, Surrey (CA); Genc Basha, Vacouver (CA); Kyla Omilusik, Vancouver (CA); Ana Chavez-Steenbock, Vancouver (CA)

(72) Inventors: Wilfred Jefferies, Surrey (CA); Genc Basha, Vacouver (CA); Kyla Omilusik, Vancouver (CA); Ana Chavez-Steenbock, Vancouver (CA)

(73) Assignee: bioMmune Technologies Inc., Victoria, BC (CA)

(21) Appl. No.: 14/373,841

(22) PCT Filed: Jul. 31, 2012
(86) PCT No.: PCT/CA2012/050519

§ 371 Date: May 15, 2015

Related U.S. Application Data
(60) Provisional application No. 61/591,415, filed on Jan. 27, 2012.
(60) Provisional application No. 61/594,863, filed on Feb. 3, 2012.

(51) Int. Cl. A61K 39/205; A61K 38/17; A61K 35/15; C12N 7/00; C07K 14/005; C07K 14/705

(52) U.S. Cl. CPC A61K 39/205; C07K 14/005; C07K 14/705; A61K 35/15; C12N 7/00; A61K 38/177; C07K 2319/06; C12N 2760/20234

(57) ABSTRACT

Compositions for and methods of stimulating a MHC I mediated immune response comprising stimulating MHC I endolysosomal cross presentation in dendritic cells. Stimulation MHC I endolysosomal cross presentation may comprise over-expression CD74 in dendritic cells and/or targeting antigens to the MHC I endolysosomal cross presentation pathway. Fusion proteins comprising an antigen or fragment thereof and a CD74 endolysosomal targeting sequence are also provided.

### FIELD OF THE INVENTION

The present invention relates to the field of immune modulation, in particular, compositions and methods of modulating of MHC I mediated immune responses.

### BACKGROUND

During primary immune responses, dendritic cells are the principal antigen-presenting cells (APCs) that initiate adaptive immune responses. Dendritic cells take up dead cells and cellular debris containing antigenic proteins and process these exogenously-derived antigens for presentation on MHC I. This process is referred to as MHC I cross-presentation. This process is essential for CD8⁺ T cell mediated responses against viruses, tumours, self antigens and allografts.

CD74 is an important piece of cellular machinery working inside dendritic cells to regulate the mammalian primary immune response. Dendritic cells possess specialized pathways that enable them to sense and then respond to foreign threats. Until now no one has been able to piece together the circuitry which enables Major Histocompatability Class I (MHC I) to find and ‘collide’ with foreign invaders resulting in the essential presentation and recognition of pathogens by the immune system.

### SUMMARY OF THE INVENTION

An object of the present invention is to provide compositions and methods of modulating an immune response. In accordance with one aspect of the present invention, there is provided a method of stimulating a MHC I mediated immune response comprising stimulating MHC I endolysosomal cross presentation in dendritic cells. Stimulating MHC I endolysosomal cross presentation may comprise over-expressing CD74 in dendritic cells and/or targeting antigens to the MHC I endolysosomal cross presentation pathway.

In accordance with another aspect of the present invention, there is provided a fusion protein comprising an antigen or fragment thereof and a CD74 endolysosomal targeting sequence. Nucleic acid molecules, vectors and cells expressing the fusion protein are also provided.

In accordance with another aspect of the present invention, there is provided a compartment for CD74-dependent MHC I cross presentation pathway. This compartment may be an endolysosome.

In accordance with another aspect of the present invention, there is provided a cathepsin cleaved peptide and concatemers of said peptides for stimulating primary immune response.

### BRIEF DESCRIPTION OF THE FIGURES

FIG. 1. Cd74^{-/-} mice generate weak antiviral primary immune responses. (a) Generation of VSVNP(52-59)-specific (H-2K^{b}-VSVNP) CD8⁺ T cells in spleens of Cd74^{+/+}, Cd74^{-/-} and Tap1^{-/-} mice isolated 6 d after infection with a low titer of VSV (2×10⁵ of a dose that infects 50% of a tissue culture cell monolayer or mouse), then stimulated for 5 d with VSVNP(52-59). Numbers in quadrants indicate percent cells in each throughout. (b) Frequency of H-2K^{b}-VSVNP(52-59)-specific CD8⁺ T cells among cells obtained as in a (n=3 mice/genotype). (c) Standard ⁵¹Cr-release assays of CTLs generated after VSV infection and in vitro boosting as in a. *P<0.05 (Student's t-test). Data are representative of at least 3 separate experiments (mean±s.d.).

FIG. 2. The deficiency of Cd74′ mice in eliciting primary immune responses resides in their antigen-presenting cells and is independent of CD4⁺ T cells. (a) Generation of VSVNP(52-59)-specific CD8⁺ cells in spleens obtained from chimeras (n=3) injected with VSV (1×10⁵ of a dose that infects 50% of a tissue culture cell monolayer or mouse) and boosted in vitro with VSVNP(52-59). (b) Cytotoxicity assays of CTLs generated after in vitro boosting as in a. Target cells were pulsed with VSVNP(52-59) where indicated or left unlabeled as a control for non-specific killing. (c) Generation of CD8⁺ T cells specific for H-2K^{b}-VSVNP(52-59) in the spleens of Cd74^{+/+}→Cd74^{-/-} and Cd74^{-/-}→Cd74^{+/+} chimeras (n=3) depleted of CD4⁺ cells by intravenous injection of anti-CD4 (+Ab) following VSV infection and in vitro boosting as in a. (d) Cytotoxicity assays as in b of CTLs generated after in vitro boosting as in c. *P<0.05 (Student's t-test). Data are representative of 3 experiments (a), at least 3 experiments (b), 2 experiments (c) or 3 experiments (d; mean and s.d.).

FIG. 3. Cd74^{-/-} DCs are unable to cross-present cell-associated antigens in vivo to prime antigen-specific CD8⁺ T cells. (a) Protocol: OVA protein or OVA(257-264) pulsed Cd74^{-/-} or Cd74^{+/+} BMDCs were injected into Rag1^{-/-} mice on a BALB/c background, along with purified CFSE-labeled CD8⁺ OT-I T cells (left); 3 d later, the proliferation of H-2K^{b} CD8⁺ T cells (outlined area, right) was assessed. (b) Proliferating (black) and non-proliferating (gray) OT-I T cells from the spleens of the recipient mice in a (n=3). Numbers above bracketed lines indicate percent CFSE⁻ (dividing) cells. Data are representative of 2 experiments.

FIG. 4. Cross-presentation and cross-priming are defective in Cd74^{-/-}DCs. (a) Uptake of OVA Alexa Fluor 488 by BMDCs was assessed by flow cytometry after incubation with OVA at 37° C. (dark gray shaded curve) or 4° C. (light gray line). (b) Formation of H-2K^{b}-OVA(257-264) complexes on splenic DCs with (+OVA) or without (−OVA) incubation with soluble OVA (top), as well as total H-2K^{b} (shaded curve) above background (gray line; bottom), measured by flow cytometry. (c) Expression of CD80 and CD40 on BMDCs incubated with medium alone (−OVA), OVA alone (+OVA) or OVA and interferon-γ (OVA+IFN-γ), assessed by flow cytometry. (d) Activation of B3Z T cells by spleen-derived DCs incubated with various concentrations of soluble OVA (horizontal axis) in the presence of the cell-signaling molecule GM-CSF alone (top) or GM-CSF plus TNF (middle) or interferon-γ (bottom), measured by chemiluminescence assay. (e) ICM of mature, spleen-derived DCs incubated with OVA with (bottom) or without (top) TNF, then costained with antibody to H-2K^{b}-OVA(257-264) (red) and anti-LAMP-1 (green), presented as optically merged images. Scale bar, 5 μm. (f) Quantitative analysis of the colocalization of H-2K^{b}-OVA(257-264) with LAMP-1⁺ late endosomes in the presence of TNF (top) and fluorescence of Cd74^{+/+}, Cd74^{-/-} and Tap1^{-/-} DCs (>20 per strain) with and without TNF treatment (bottom), presented as normalized individual pixels relative to total pixels. *P<0.05 (Student's t-test). Data are representative of 2 (a-f) experiments (error bars (f), s.d.) or are from 1 experiment representative of 3 separate experiments with similar results (d; mean±s.d. of triplicate samples).

FIG. 5. Inhibition of CD74-mediated trafficking of MHC class I in DCs by treatment with chloroquine. (a) Formation of H-2K^{b}-OVA(257-264) complexes on BMDCs left untreated (−CQ) or treated with chloroquine (+CQ) and incubated with medium alone (blue) or with soluble OVA (red; top) or OVA peptide (red; bottom), measured by flow cytometry. (b) Total H-2K^{b} (green) on BMDCs left untreated or treated with chloroquine; blue, background. (c) Surface H-2K^{b}-OVA(257-264) complexes on BMDCs treated as in a, presented as normalized mean fluorescence intensity (MFI) where 100% is the amount of H-2K^{b}-OVA(257-264) complexes found on untreated BMDCs. (d) ICM of mature BMDCs left untreated or treated with chloroquine, then costained with anti-H-2K^{b} (red) and anti-CD74 (green), presented as optically merged images. Scale bar, 5 μm. (e) Quantification of the colocalization of H-2K^{b} with CD74 in d, presented as normalized pixels relative to total pixels. (f) Proliferation of CFSE-labeled OT-I cells induced by Cd74^{-/-} BMDCs reconstituted with full-length (+FL) CD74 or truncated CD74 lacking the endolysosomal trafficking motif (+Δ2-17) and incubated with soluble OVA protein or OVA(257-264). Numbers in outlined areas indicate percent proliferating OT-I cells (CD8⁺CFSE⁻) relative to that of Cd74^{+/+} control (far left), set as 100%. Data are representative of 2 experiments (error bars (c,e), s.d.).

FIG. 6. CD74 controls ER-to-endolysosome trafficking of MHC class I in DCs. (a) ICM of mature splenic DCs stained with anti-H-2K^{b} (green) plus anti-CD74 (red; top) or anti-LAMP-1 (red; bottom). Scale bar, 5 μm. (b) Quantification of MHC class I in LAMP-1⁺ compartments (50 DCs per mouse strain), presented as individual pixels/total pixels. (c) Immunoprecipitation (IP) of [³⁵S]methionine-labeled Cd74^{+/+}, Cd74^{-/-}, Tap1^{-/-} and β₂-microglobulin-deficient (B2m^{-/-}) BMDCs with anti-I-A-I-E (I-A^{b}; left lane), anti-CD74 (middle lane) or anti-H-2K^{b} (right lane). Arrows indicate 41-kDa (top) and 31-kDa (bottom) CD74 bands. (d) Immunoprecipitation of proteins from lysates of Cd74^{+/+} DCs with anti-I-A^{b}, anti-H-2K^{b} (conformationally dependent), antibody to the H-2K^{b} cytoplasmic domain (e-VIII; conformationally independent) or antibody to the transferrin receptor (TFR), followed by immunoblot analysis with anti-CD74. Far right (WCL), immunoblot analysis of whole-cell lysates (control). (e) Immunoprecipitation of proteins from lysates of DCs with anti-CD74, followed by no digestion (−) or digestion with Endo H (+) and immunoblot analysis with anti-MHC class I. (f) Immunoprecipitation, with anti-MHC class I, of proteins from lysates of cells left untreated or treated with chloroquine or Endo H, followed by immunoblot analysis with anti-CD74. Band intensities were quantified using the Odyssey software. Numbers below the lanes indicate the band intensity normalized to CQ untreated samples (g) Internalization of MHC class I in DCs labeled with anti-H-2K^{b}, evaluated over time by flow cytometry and presented as the percent decrease in mean fluorescence intensity of DCs incubated at 37° C. compared to the control DCs at 4° C. *P<0.05 (Student's t-test). Data are representative of 2 experiments (a), 2 experiments (b; mean±s.d.), 5 experiments (c), 3 experiments (d), 3 experiments (e), 2 experiments (e) or 2 experiments (f; error bars, s.d.).

FIG. 7. Peripheral Analysis of Chimeric Mice.

FIG. 8. CD74^{-/-} mice are unable to cross-present cell-associated antigens in vivo to generate an effective primary immune response.

FIG. 9. CD74^{-/-} DCs localize to the spleen.

### DESCRIPTION

The present invention is based on the discovery of the guiding role played by CD74 to link MHC I receptors to compartments containing invading pathogens within the immune cell. This sophisticated circuit allows the immune cell to recognize and signal the presence of a pathogen in the body and to alert specialized T immune fighter cells which respond by dividing, and attacking infected cells, thereby destroying the pathogen. In particular, the present invention is based on the discovery that CD74 mediates trafficking of MHC I from the endoplasmic reticulum of dendritic cells to endolysosomal compartments for loading with exogenous peptides and therefore CD74 has a critical function in endolysosomal dendritic cell cross-presentation for priming MHC I mediated CTL responses. Accordingly, the present invention provides methods of modulating MHC I mediated immune responses.

In certain embodiments, there is provided compounds and methods of modulating CD74 dependent MHC I endolysosomal dendritic cell cross-presentation. In certain embodiments, there is provided a method of stimulating an immune response, such as a MHC I mediated CTL response, by enhancing CD74 dependent MHC I dendritic cell cross-presentation. The CD74 dependent MHC I cross-presentation pathway may be enhanced, for example, by increasing expression of CD74 in dendritic cells. Accordingly, in certain embodiments there are provided compounds and methods to enhance expression of CD74.

Expression vectors may be used to express a CD74 protein of the present invention in cells. Appropriate expression vectors which may be used in the construction of an expression vector would be apparent to a worker skilled in the art. It would also be apparent to a worker skilled in the art that such vectors may be administered directly to an individual. Alternatively cells from an individual may be engineered with a polynucleotide (DNA or RNA) encoding a polypeptide of the invention ex vivo, with the engineered cells then being provided to the individual. Such methods are well-known in the art.

Furthermore, the amino acid sequence of murine CD74 is known in the art (see, for example, NCBI Protein database Accession No. P04441.3) and is set forth below:

| | |
|---|---|
| 1 | mddqrdlisn heqlpilgnr prepercsrg alytgvsvlv |
| | alllagqatt ayflyqqqgr |
| 61 | ldkltitsqn lqleslrmkl pksakpvsqm rmatpllmrp |
| | msmdnmllgp vknvtkygnm |
| 121 | tqdhvmhllt rsgpleypql kgtfpenlkh lknsmdgvnw |
| | kifeswmkqw llfemsknsl |
| 181 | eekkpteapp kvltkcqeev shipavypga frpkcdengn |
| | ylplqchgst gycwcvfpng |
| 241 | tevphtksrg rhncsepldm edlssglgvt rqelgqvtl |

The amino acid sequence of various isoforms of human CD74 are also known in the art (see, for example, Genbank Accession numbers AAH18726.1; AAH24272.1; EAW61729.1; EAW61730.1 and EAW61731.1).

Accordingly, in certain embodiments of the invention, there is provided polynucleotides and expression vectors which express CD74 and methods of utilizing such polynucleotides and expression vectors to express of CD74 or active fragments thereof. In certain embodiments, the polynucleotides and expression vectors of the invention are used to genetically engineer cells, including but not limited to dendritic cells, in vivo. In certain other embodiments, the polynucleotides and expression vectors of the invention are used to genetically engineer cells, including but not limited to dendritic cells, ex vivo and these genetically engineered cells may then be administered to the individual. Accordingly, in certain embodiments, there is provided dendritic cells which have been genetically engineered to over-express CD74. The polynucleotides, expression vectors and cells may be administered as a pharmaceutical composition with a pharmaceutically acceptable diluent or carrier.

As noted above, there is evidence to suggest that the endolysosome is the principal compartment for cross-presentation in dendritic cells. In certain embodiments of the invention, there is provided the endolysosome of the dendritic cell. In certain embodiments, there is provided the peptides for presentation to MHC I generated in the endolysosomal compartment of a dendritic cell. These peptides may be peptides processed from antigens and fragments thereof, specifically targeted to the endolysosome of the dendritic cell.

Targeting antigens and fragments thereof to the endolysosomal compartment of dendritic cells may enhance priming of MHC I antigens. Accordingly, in certain embodiments of the invention, there are provided compounds and methods to target molecules, including antigens and fragments thereof, to the endolysosome of dendritic cells. For example, the endosomal targeting signal of CD74 may be used to route antigens or fragments thereof to the MHC I antigen processing pathway in dendritic cells. In certain embodiments of the invention, there is provided fusion proteins comprising an antigen of interest, or fragment thereof, and the CD74 endosomal targeting signal. In certain embodiments, the targeting signal comprises amino acids 2 to 17 of the sequence set forth in NCBI Protein database Accession No. P04441 (sequence: ddqrdlisn heqlpil).

Polynucleotides, expression vectors and cells (including dendritic cells) expressing the fusion proteins of the invention are also provided. As noted above, appropriate expression vectors would be apparent to a worker skilled in the art. The polynucleotides and expression vectors expressing the fusion protein may be used to genetically engineer cells, including but not limited to dendritic cells, in vivo or may be used to genetically engineer cells, including but not limited to dendritic cells, ex vivo and these genetically engineered cells may then be administered to the patient. The fusion proteins, polynucleotides, expression vectors and cells may be administered as a pharmaceutical composition with a pharmaceutically acceptable diluent or carrier.

Enhancement of MHC I cross-presentation may result in enhancement of an immune response. In particular, enhancement of CD74 dependent MHC I endolysosomal dendritic cell cross-presentation may result in stimulation of a MHC I mediated CTL response. Accordingly, in certain embodiments of the invention, there is provided a method of stimulating a MHC I mediated CTL response by enhancing MHC I endolysosomal cross-presentation. As noted above, enhancement of MHC I cross-presentation may be through over-expression of CD74 and/or targeting antigens or fragments thereof to the MHC I antigen processing pathway in dendritic cells. These methods may be combined with other immunostimulatory methods, such as administration of immunostimulatory compounds, including but not limited to cytokines, to further stimulate an immune response. Other immunostimulatory methods and compounds appropriate for use with the compounds and methods of the present invention would be apparent to a worker skilled in the art.

A worker skilled in the art would readily appreciate that stimulation of a CTL response may be useful in the prevention and/or treatment of a number of diseases and/or conditions. For example, stimulation of a CTL response may be useful in the prevention and/or treatment of diseases caused by intracellular pathogens including but not limited to bacteria, plasmodium and viruses, and/or treatment of cancer. Accordingly, in certain embodiments of the invention, there is provided methods of preventing and/or treating diseases caused by intracellular pathogens by stimulating the MHC I cross-presentation pathway. In other embodiments, there is provided methods of treating cancer by stimulating the MHC I cross-presentation pathway.

In certain embodiments, there is provided a method of preventing and/or treating viral infections, including but not limited to HIV infection. In certain embodiments, there is provided a method of preventing and/or treating bacterial infections, such as mycobacterial infections including but not limited to M. tuberculosis infections. In certain embodiments, there is provided a method of preventing and/or treating plasmodium infections, including but not limited to prevention and/or treatment of malaria.

Compounds and methods which enhance priming for MHC I antigens may be useful in improving the immunogenicity and efficacy of vaccines. Accordingly, the compounds of the invention may be used as adjuvants and/or vaccines. For example, polynucleotides, expression vectors and/or dendritic cells which express CD74 may be used to stimulate an immune response. In addition, fusion proteins (and polynucleotides and/or expression vectors expressing the fusion protein) which target the MHC I cross-presentation pathway may be used in vaccines. Accordingly, in certain embodiments, there is provided vaccines which target the MHC I cross-presentation pathway.

In certain embodiments, there is provided cathepsin cleaved peptides for stimulating primary immune responses in vaccines and concatemers of these peptides. In certain embodiments, the cathepsin is Cathepsin S.

In certain embodiments, there is provided compounds and methods for improving performance of a vaccine. In certain embodiments, there is provided compounds and methods for improving performance of a cancer vaccine. In certain embodiments there is provided compounds and methods for improving performance of a vaccine against a virus, including but not limited to HIV. In certain embodiments there is provided compounds and methods for improving performance of a vaccine against a bacteria, such as mycobacteria including but not limited to M. tuberculosis. In certain embodiments, there is provided compounds and methods for improving performance of a vaccine against plasmodium, including but not limited to Plasmodium falciparum.

An understanding of the role of CD74 may also begin to explain differences in immune responses between individuals that could impact personalized medical options in the future. Accordingly, in certain embodiments, there is provided a method of developing personalized vaccine approaches based on an individual's CD74-dependent MHCI cross-presentation pathway.

Inhibition of MHC I cross-presentation may result in inhibition of an immune response. For example, deficiencies in CD74 expression may result in a decrease in MHC I cross-presentation which in turn may decrease MHC I mediated immune responses, including MHC I mediated CTL responses. Accordingly, in certain embodiments of the invention, there is provided methods of inhibiting MHC I cross-presentation and thereby inhibiting MHC I mediated immune responses by inhibiting the expression and/or activity of CD74 in dendritic cells. Such methods may be useful in the treatment of autoimmune diseases and/or the prevention/inhibition of graft rejection. Compounds which inhibit the expression and/or activity of CD74 may include, for example, antisense compounds and/or neutralizing antibodies.

It has been suggested that MHCI signaling may effect Toll-like receptor (TLR) innate inflammatory responses. In particular, it was found that constitutively expressed membrane MHC I attenuated TLR-triggered innate inflammatory responses. (Nature Immunology 13: 551-559). Accordingly, in certain embodiments of the invention, there is provided methods of modifying the innate immune response by modifying MHCI signaling.

The effect of the compounds of the invention on an immune response may be tested in in vivo animal models. For example, immune responses may be assessed in vivo by reconstituting antigen presenting cells and T cells in a RAG−/− immune deficient mice. Accordingly, in certain embodiments of the present invention, there is provided methods of screening immune modulators and/or adjuvants in RAG−/− immune deficient mice comprising reconstituting the mice with dendritic cells and CD8⁺ T cells and analyzing the immune response in the mice. The candidate immune modulators may be administered directly to the mice after reconstitution and/or to the dendritic cells and/or T cells prior to injection into the RAG−/− mice.

CD74 deficient mice and/or dendritic cells may also be used in the development of vaccines which target the MHC I cross presentation pathway. For example, such mice and cells may be useful in the identification of peptides which are cross-presented by MHC I and therefore may be useful in the stimulation of a primary immune response. To gain a better understanding of the invention described herein, the following examples are set forth. It will be understood that these examples are intended to describe illustrative embodiments of the invention and are not intended to limit the scope of the invention in any way.

### Example

### A CD74-Dependent MHC Class I Endolysosomal Cross-Presentation Pathway

Immune responses are initiated and primed by dendritic cells (DCs) that cross-present exogenous antigen. The chaperone CD74 (invariant chain) is thought to promote DC priming exclusively in the context of major histocompatibility complex (MHC) class II. However, here a CD74-dependent MHC class I cross-presentation pathway in DCs that had a major role in the generation of MHC class I-restricted, cytolytic T lymphocyte (al) responses to viral protein and cell-associated antigens is demonstrated. CD74 associated with MHC class I in the endoplasmic reticulum of DCs and mediated the trafficking of MHC class I to endolysosomal compartments for loading with exogenous peptides. It is concluded that CD74 has a previously undiscovered physiological function in endolysosomal DC cross-presentation for priming MHC class I-mediated CTL responses.

During primary immune responses, dendritic cells (DCs) are the principal antigen-presenting cells that initiate adaptive immune responses predominantly through cross-presentation and the cross-priming of T cells. This involves extracellular antigen uptake, digestion of cell-associated antigenic fragments and presentation of proteolytic peptide products on both major histocompatibility complex (MHC) class I and MHC class II molecules¹. For MHC class I, two main pathways have been described that may explain how this process occurs: the cytosolic pathway²⁻⁵ shown convincingly to function in vitro, and the vacuolar pathway shown to have a major role in vivo for certain antigens⁶⁻⁸. The ‘phago-ER’ (endoplasmic reticulum-mediated phagocytosis) model of cross-presentation has been considered a dominant pathway of cross-presentation⁹. Subsequent data have disputed that conclusion¹⁰. One factor that has contributed to this controversy seems to be the over-interpretation of data that designate intracellular proteins as definitive markers of specific organelles that are often not exclusive but merely undergo enrichment during dynamic organelle biogenesis and partitioning. Furthermore, contrasting conclusions may have been inferred from studies of different forms of exogenous antigens and in studies of long-term DC cell lines versus those of freshly isolate DCs.

In the vacuolar pathway, cathepsin S has been identified as a protease that generates antigenic peptides that are loaded onto peptide-receptive MHC class I molecules¹¹. Furthermore, membrane and cytosolic SNARE proteins, which control tethering and docking events for donors and acceptors during intracellular membrane fusion, also seem to have a fundamental role in cross-presentation events¹². However, the source of MHC class I in the cross-priming compartment, the mechanism of its transport and the site of peptide loading remain areas of active study^{8,13}.

Spontaneous internalization of recycling MHC class I into endosomes has been demonstrated^{14,15}. Published results support a model in which the recycling of MHC class I from the plasma membrane to an endolysosomal loading compartment is facilitated by recognition of the tyrosine internalization signal found in the MHC class I cytoplasmic tail^{8,13}. Therefore, MHC class I molecules recycling from the plasma membrane is one source of MHC class I for loading with exogenous antigens destined for participation in cross-presentation^{8,13}. Likewise, transport of MHC class I from the endoplasmic reticulum (ER) to the endocytic compartment has also been proposed. This could occur by a mechanism involving fusion of the phagosome and ER⁹. An alternative and potentially complementary hypothesis is that chaperone CD74 (invariant chain), known to associate with MHC class II in the ER, thereby preventing premature binding of peptides and mediating trafficking to the endocytic pathway by sorting signals present in the CD74 cytoplasmic tail^{1,16}, could bind MHC class I and deliver a fraction of the MHC class I to the vacuolar-endocytic compartmentto function in cross-presentation^{17,18}. This mechanism would coincidently place peptide-receptive MHC class I in the same compartment with exogenous antigen and MHC class II molecules (or a similar compartment)¹⁹, the MIIC compartment, facilitating antigenic peptide loading and binding to MHC class I molecules. This pathway would link MHC class I transport to the vacuolar pathway, as it is unlikely that CD74 would be involved in the cytosolic route of MHC class I exogenous presentation^{20,21}.

The interaction of MHC class I with CD74 and their coincident localization in the same compartment has been demonstrated in human cell lines¹⁷⁻¹⁹. Although it was concluded on the basis of older paradigms that the MHC class I-CD74 interaction probably does not control the fate of the transport of MHC class I to endosomes under physiological conditions²², other contrasting studies have demonstrated that cells transfected to express CD74 have much higher surface expression of MHC class I encoded by diverse alleles, which suggests that the MHC class I-CD74 interaction might have functional importance²³. Here is investigated the immunological relevance of MHC class I interaction with CD74 in vivo and a clear and critical role for CD74 in the cross-presentation of exogenous antigen and subsequent cross-priming by DCs is described.

### Results

### CD74 is Required for Primary Antiviral Responses

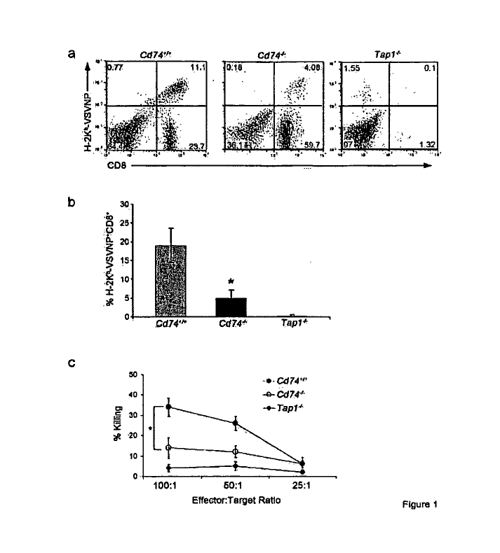

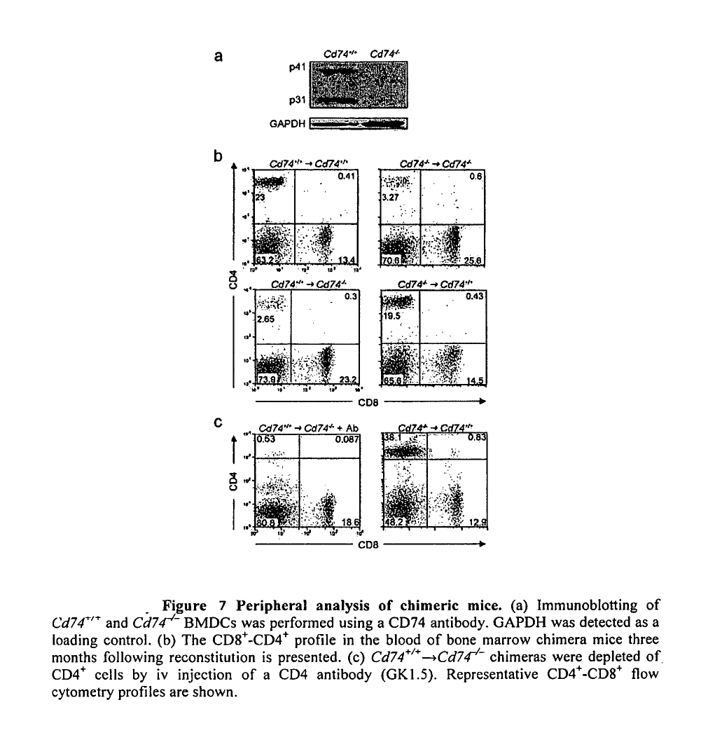

DCs can be directly infected and could therefore use classical presentation by MHC class I to activate naive CD8⁺ T cells. However, during infection with virus at a low titer, direct infection of DCs is less likely and DC cross-presentation is the dominant pathway responsible for generation of CD8⁺ T cell responses^{8,24}. To address the role of CD74 in cross-presentation to generate primary antiviral immune responses, wild-type (Cd74^{+/+}) mice and CD74-deficient (Cd74^{-/-}) mice were infected with a low dose of vesicular stomatitis virus (VSV). We similarly infected mice deficient in the transporter TAP (Tap1^{-/-} mice), which have impaired assembly and intracellular transport of MHC class I and thus lack CD8⁺ T cells due to improper thymic selection, as a negative control²⁵ (FIG. 1 and FIG. 7a). In this infection, primary and memory CD8⁺ T cell responses to VSV can be generated in the absence of CD4⁺ T cells^{26,27}. In this way, the role of CD74 in cross-presentation can be assessed regardless of its role in CD4⁺ T cell responses. The frequency of CD8⁺ T cells generated in response to the immunodominant epitope VSV nucleoprotein amino acids 52-59 (VSVNP(52-59)) presented on MHC class I (H-2K^{b}) after VSV infection was assessed. Cd74^{-/-} mice had a significantly lower capacity to generate antigen specific CD8⁺ T cells than did Cd74^{+/+} mice (5.0% versus 19.0%; FIG. 1a,b). This resulted in an immune response with less cytotoxic T lymphocyte (CTL) killing capacity (FIG. 1c).

Bone marrow chimeras were constructed to further exclude the possibility of a role for T cell help in cross-priming in the VSV infection model^{26,27}. Additionally, the chimeras were used to confirm whether the deficiency in generating immune responses was dependent on the ability of the hematopoietic cell-derived DCs to cross-present antigen and prime T cells. For this Cd74^{+/+} mice with Cd74^{+/+} bone marrow (Cd74^{+/+}→Cd74^{+/+}) or Cd74^{-/-} bone marrow (Cd74^{-/-}→Cd74^{+/+}) and reconstituted Cd74^{-/-} mice were reconstituted with Cd74^{+/+} bone marrow (Cd74^{+/+}→Cd74^{-/-}) or Cd74^{-/-} bone marrow (Cd74^{-/-}→Cd74^{-/-}). It was found that normal amounts of CD8⁺ T cells and CD4⁺ T cells in the periphery of Cd74^{+/+}→Cd74^{+/+} and Cd74^{-/-}→Cd74^{+/+} mice. However, fewer CD4⁺ T cells and somewhat more CD8⁺ T cells were found in Cd74^{-/-}→Cd74^{-/-} and Cd74^{+/+}→Cd74^{-/-} mice (FIG. 7b,c). This indicated that positive selection in recipient Cd74^{-/-} mice was impaired because of a lower abundance of MHC class II in the Cd74^{-/-} thymic epithelium.

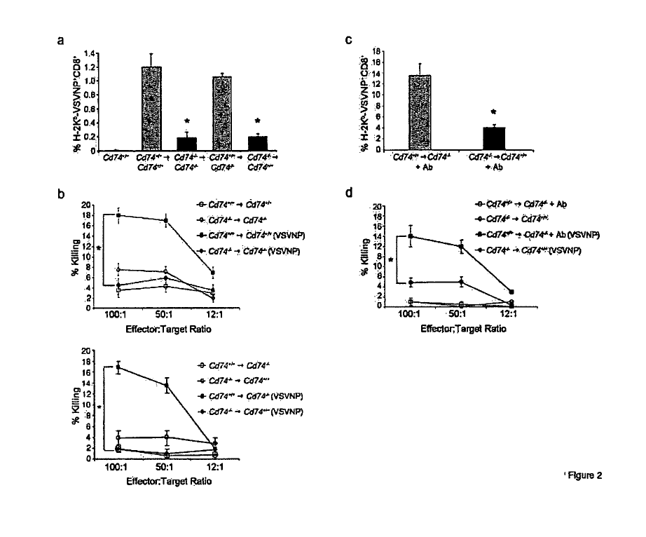

To examine antiviral responses, chimeric mice were infected with a low titer of VSV and assessed VSVNP(52-59)-specific CD8⁺ T cell generation by tetramer analysis and a CTL killing assay (FIG. 2). Cd74^{+/+}→Cd74^{-/-} mice, with low CD4⁺ T cell numbers, were able to produce VSVNP(52-59)-specific CD8⁺ T cells similar to wild-type Cd74^{+/+}→Cd74^{+/+} chimeras (1.1% versus 1.2%; FIG. 2a), which resulted in immune responses with similar killing capacity (16.8% versus 1.9%; FIG. 2b). However, Cd74^{-/-}→Cd74^{+/+} mice were grossly impaired in the generation of VSVNP(52-59)-specific CD8⁺ T cells (0.2%; FIG. 2a) despite having normal CD4⁺ T cells, which resulted in lower CTL killing responses (18.0% versus 4.5%; FIG. 2b). This suggested that the generation of VSV specific CTL responses was independent of CD4⁺ T cell numbers. Notably, bone marrow-derived antigen-presenting cells expressing CD74 were required and allowed Cd74^{-/-} mice to produce a robust antiviral immune response similar to that of Cd74^{+/+} mice.

### Depletion of CD4⁺ Cells has No Effect on Anti-VSV Responses

Next the possibility that residual CD4⁺ T cells in the Cd74^{+/+}→Cd74^{-/-} chimeras that resulted from dysfunctional positive selection in Cd74^{-/-} mice contributed to the efficiency of their antiviral immune responses was eliminated. During the course of the infection, Cd74^{+/+}→Cd74^{-/-} chimeras were depleted of the CD4⁺ cells by injecting them with the GK1.5 antibody to CD4 (anti-CD4). Although CD4⁺ cells were almost completely undetectable relative to background, Cd74^{+/+}→Cd74^{-/-} chimeras depleted of CD4⁺ cells generated significantly more CD8⁺ T cells specific for VSVNP(52-59) than did Cd74^{-/-}→Cd74^{+/+} chimeras (13.5% versus 4.1%; FIG. 2c), which resulted in an immune response with more lytic activity (14.0% versus 4.9%; FIG. 2d). Together these data confirmed that Cd74^{+/+}→Cd74^{-/-} chimeras mounted stronger anti-VSV responses than did Cd74^{-/-}→Cd74^{+/+} chimeras. This was independent of CD4⁺ T cells but was instead due to the reconstitution of Cd74^{-/-} mice with wild-type DCs that were fully able to prime antiviral CD8⁺ T cells responses.

### Cross-Priming of Cell-Associated Antigen is CD74 Dependent

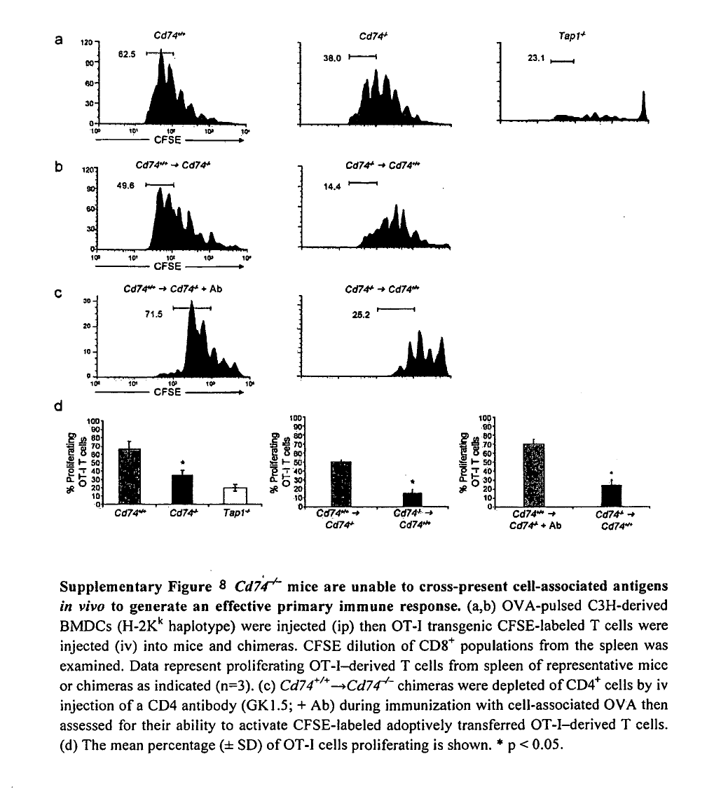

To investigate the role of CD74 in the primary immune response to cell-associated antigen, lethally irradiated DCs pulsed with ovalbumin (OVA) or DCs with MHC class I mismatched to the host pulsed with OVA as a source of cell-associated antigen to activate CTLs in Cd74^{+/+} mice, Cd74^{+/+} mice depleted of CD4⁺ cells, and Cd74^{-/-} mice, as well as in reconstituted mouse chimeras were used. Mice with a wild-type immune system, challenged with cell-associated OVA, were able to induce proliferation of CD8⁺ T cells derived from OT-I transgenic mice (FIG. 8) or activate endogenous CTLs that were efficient at killing OVA amino acids 257-264 (OVA(257-264)-pulsed target cells (data not shown). However, with the same challenge of cell-associated OVA, mice with a hematopoietic system deficient in CD74 were much less able to stimulate the proliferation of OT-I CD8⁺ T cells and generated fewer endogenous CTLs that contributed to a lower killing ability (FIG. 8 and data not shown).

### CD74-Dependent Cross-Priming is Independent of CD4⁺ T Cells

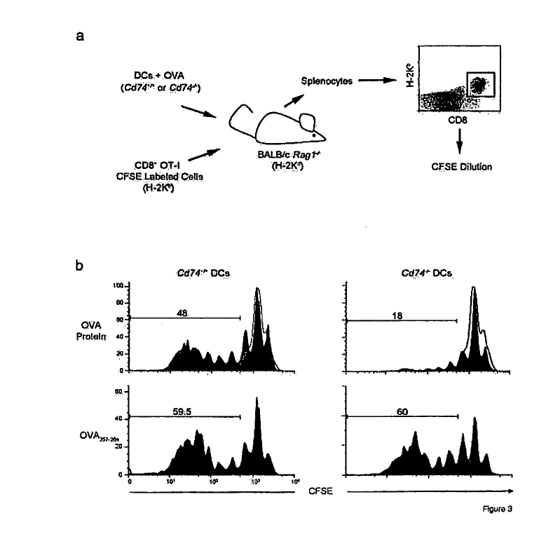

To focus specifically on DC cross-priming defects and eliminate the contribution of extraneous factors, including the requirement for CD4⁺ T cell help, Cd74^{-/-} and Cd74^{+/+} DCs were incubated with OVA protein or OVA(257-264) peptide and injected those cells along with purified OT-I CD8⁺ T cells labeled with the cytosolic dye CFSE into T cell-deficient recombination-activating gene 1 (Rag1^{-/-}) mice on a BALB/c background. The ability of the DCs to cross-prime the OT-I T cells was assessed (FIG. 3a). Cd74^{-/-} DCs induced much less OT-I proliferation than did Cd74^{+/+} DCs when incubated with OVA protein (18% versus 48%; FIG. 3b). However, when the DCs were pulsed with OVA(257-264) peptide, as a positive control for direct presentation, Cd74^{-/-} DCs were as competent as Cd74^{+/+} DCs in activating the CD8⁺ OT-I T cells (59.5% versus 60.0%; FIG. 3b).

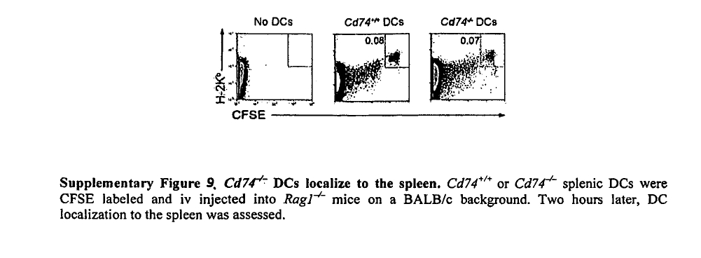

To address the possible confounding role of CD74 in the motility and homing of DCs²⁸ from the site of injection to the spleen, the localization of CFSE-labeled DCs after intravenous injection²⁹ was assessed (FIG. 3b and FIG. 9). Cd74^{+/+} and Cd74^{-/-} DCs injected intravenously into Rag1^{-/-} mice localized equivalently to the spleen. Therefore, the lower ability of Cd74^{-/-} DCs to induce T cell proliferation was not due to differences in DC migration but was due to less ability to process and present antigen. It was concluded that CD74 has a critical role in cross-presentation of cell-associated antigen by MHC class I and in CD8⁺ T cell priming in vivo and this is unrelated to CD4⁺ T cell help or CD74-mediated motility of DCs.

CD74-Deficient DCs have Impaired Cross-Priming Ability

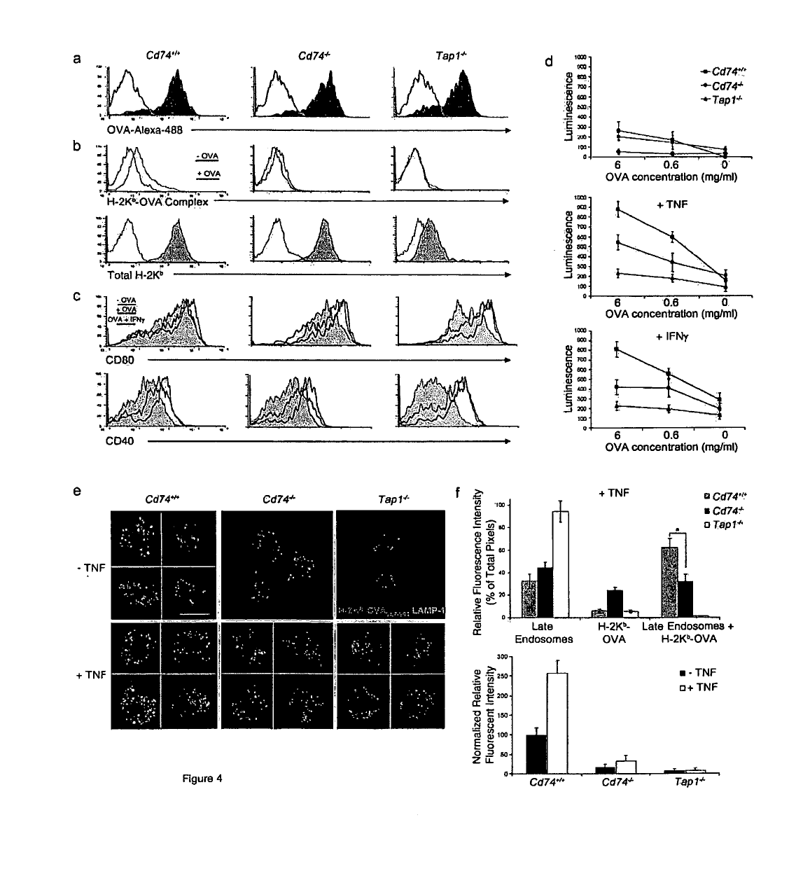

The ability of spleen-derived DCs from various mouse strains to cross-present the H-2K^{b}-restricted ovalbumin epitope OVA(257-264) in vitro was assessed. DCs were incubated with soluble OVA, with or without cytokines, and either stained the cells with an antibody specific for the H-2K^{b}-OVA(257 complex or cultured the cells with B3Z, a T cell hybridoma that is activated after recognition of H-2K^{b} in association with OVA(257-264)³⁰. Cd74^{+/+} and Cd74^{-/-} DCs had similar ability to internalize OVA and had similar total surface expression of MHC class I (FIG. 4a,b). However, after incubation with OVA, Cd74^{-/-} DCs had a much lower abundance of H-2K^{b}-OVA(257-264) complexes than did Cd74^{+/+} DCs (FIG. 4b). It has been shown that the cross-priming ability of DCs is augmented by inflammatory mediators that induce the upregulation of costimulatory and MHC molecules and diminish endocytosis^{31,32}. This results in a greater capacity for T cell priming but diminished ability of DCs to capture and present soluble antigens. To assess T cell activation in a situation resembling in vivo conditions that involves costimulation, OVA-pulsed DCs incubated with B3Z T cells with and without cytokines. In the presence of tumor necrosis factor (TNF) and interferon-γ, Cd74^{+/+} and Cd74^{-/-} DCs had an equal ability to upregulate the costimulatory molecules CD80, CD86, and CD40 (FIG. 4c and data not shown), but Cd74^{-/-} DCs were much less able to activate B3Z T cells than were Cd74^{+/+} DCs (FIG. 4d). As expected, no T cell activation was detected after the cells were incubated with OVA-pulsed DCs derived from Tap1^{-/-} mice in the presence of cytokines. These data supported the conclusion that CD74 has a role in T cell cross-priming and does not affect the expression of costimulatory molecules.

### CD74 Mediates Endolysosomal MHC Class I Loading

To better understand the mechanism of the cross-presentation and priming deficiency at a molecular level, comparative immunofluorescence confocal microscopy (ICM) was used to assess the intracellular localization, trafficking and distribution of OVA(257-264)-loaded MHC class I in Cd74^{+/+} and Cd74^{-/-} DCs with and without TNF. Cells were incubated with OVA protein and stained cells intracellularly with antibody to H-2K^{b}-OVA(257-264) and to the late endosome marker LAMP-1. Colocalization with LAMP-1 was detectable in many of the Cd74^{+/+} splenic DCs that stained for H-2K^{b}-OVA(257-264) complexes when no TNF was added to the culture (FIG. 4e,f). Some H-2K^{b}-OVA(257-264) complexes in the Cd74^{-/-} and Tap1^{-/-} DCs were identified; however, colocalization with late endosomes was minimal (FIG. 4e,f). The absence of loaded MHC class I in the Tap1^{-/-} DCs was consistent with a role for TAP in cross-presentation, a mechanism that has been postulated before^{24,33}. After treatment with TNF, Cd74^{+/+} DCs had significantly more colocalization of H-2K^{b}-OVA(257-264) complexes with LAMP-1 (FIG. 4e,f) but not with the ER marker GRP78 or the Golgi marker giantin (data not shown). In contrast, few H-2K^{b}-OVA(257-264) complexes in late endosomal compartments in Cd74^{-/-} DCs were observed which indicated less formation of H-2K^{b}-OVA(257-264) complexes in late endosomes (FIG. 4f). Comparison of the ICM data indicated that in the presence of TNF, DCs derived from Cd74^{-/-} had significantly less OVA(257-264) loaded onto H-2K^{b} in the late endosomes than did Cd74^{+/+} DCs (62% versus 32%; FIG. 4f). These data suggested that in DCs, a CD74-dependent MHC class I antigen-processing pathway exists that is required for the cross-presentation of exogenous antigens.

CD74 Directs MHC Class I from the ER to the Endolysosomes

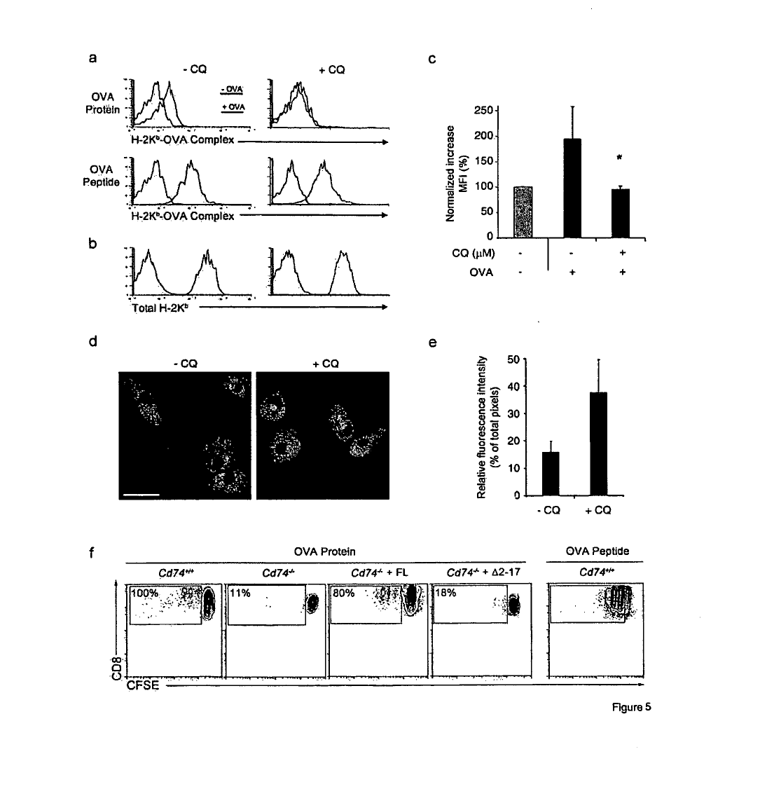

The finding that CD74 deficiency resulted in fewer H-2K^{b}-OVA(257-264) complexes in late endosomal compartments suggested that CD74 targets MHC class I from the ER to the endolysosomal pathway. There, CD74 is presumably degraded and MHC class I is loaded with exogenous antigenic peptides. To examine this in more detail, the acidification of endosomes was blocked through the use of chloroquine and assessed the CD74-mediated MHC class I cross-presentation pathway. It was found that bone marrow-derived DC (BMDCs) treated with chloroquine had surface expression of MHC class I equivalent to that of untreated controls and displayed H-2K^{b}-OVA(257-264) when pulsed with OVA(257-264) peptide; however, when incubated with soluble OVA, chloroquine-treated DCs had much less surface H-2K^{b}-OVA(257-264) than untreated DCs (FIG. 5a-c). ICM analysis showed that BMDCs had more colocalization of H-2K^{b} and CD74 after treatment with chloroquine (FIG. 5d,e). This indicated that treatment with chloroquine resulted in more endolysosomal MHC class I molecules, presumably by blocking the dissociation of MHC class I and CD74 in the endolysosomes in a manner similar to that reported for the MHC class II pathway³⁴ and by inhibiting the degradation of recycling MHC class I. The end result was less loading of MHC class I with exogenous antigen and subsequently less surface H-2K^{b}-OVA(257-264). To confirm the finding that CD74 directed MHC class I to an endolysosomal compartment and to unequivocally demonstrate that CD74 mediated MHC class I trafficking, CD74-deficient BMDCs were transfected with expression vectors for full-length CD74 or CD74 lacking the cytosolic trafficking domain and assessed their ability to present OVA protein or OVA(257-264) peptide, a positive control that would bypass the need for processing. Cd74^{-/-} DCs had impaired cross-priming ability and induced much less OT-I T cell proliferation than did Cd74^{+/+} DCs (FIG. 5f). As expected, cross-priming ability was restored n Cd74^{-/-} DCs reconstituted with full length CD74 and DCs were able to induce OT-I T cell proliferation with an ability similar to that of wild-type DCs. However, when we reintroduced CD74 lacking the endosomal trafficking motif into cd74^{-/-} DCs, cross-priming ability continued to be impaired (FIG. 5f), which demonstrates that in the absence of CD74, there was less MHC class I directed to endolysosome and less cross-priming of OT-I T cells. Together these data showed that CD74 influenced the trafficking of MHC class I to the cross-priming compartment where efficient presentation of exogenous antigen takes place.

### CD74 and MHC Class I Molecules Form a Complex in DCs

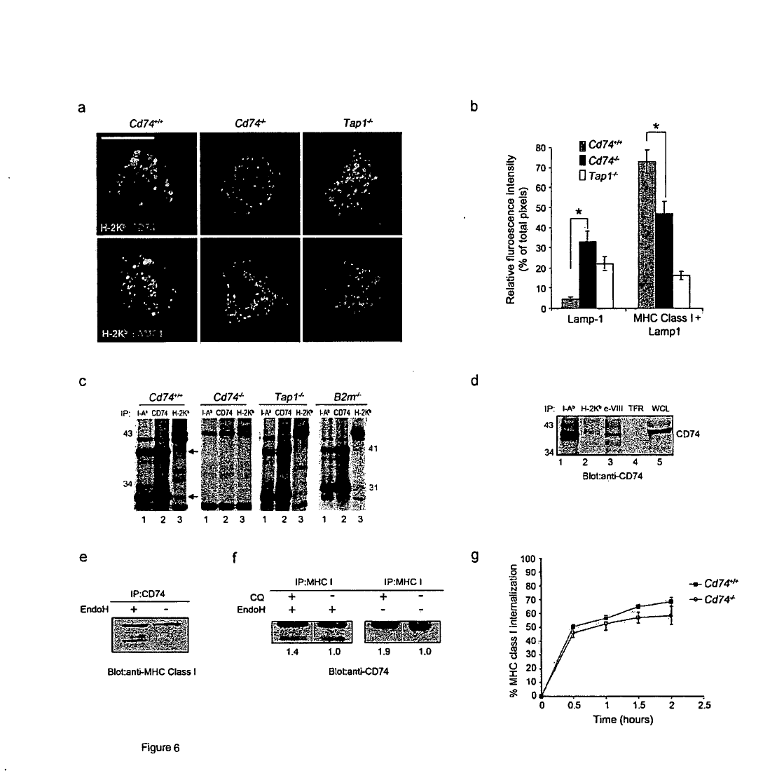

The interaction of CD74 with MHC class I in DCs as a prerequisite for the targeting of MHC class I to the cross-priming compartment was investigated at the molecular level. DCs derived from Cd74^{+/+} and Cd74^{-/-} mouse spleens were isolated for analysis by ICM. DCs were stained with anti-H-2K^{b} and anti-CD74 and found H-2K^{b} molecules were distributed at the cell surface and in the cytoplasm where they localized mainly to vesicular-like compartments. CD74 molecules colocalized considerably with these intracellular compartments in Cd74^{+/+} DCs (FIG. 6a). However, we observed less colocalization of H-2K^{b} with CD74 in Tap1^{-/-} DCs, presumably due to the restricted overall availability of H-2K^{b} (FIG. 6a).

To identify the compartment where these molecules colocalize, spleen DCs were stained with anti-H-2K^{b} and anti-LAMP-1 (to detect late endosomes). A considerable proportion of late endosomes contained H-2K^{b} in Cd74^{+/+} DCs (FIG. 6a), which confirmed that a substantial amount of MHC class I molecules reside in the endocytic compartment^{8,21}. In contrast, only a small fraction H-2K^{b} colocalized with late endosomes in Cd74^{-/-} DCs (FIG. 6a). This result was confirmed by quantification of ICM images, which suggested that significantly fewer MHC class I molecules were targeted to the endolysosomal compartment in Cd74^{-/-} DCs than in Cd74^{+/+} DCs (73% versus 47%; FIG. 6b). Colocalization was even less evident in the Tap1^{-/-} DCs, possibly due to the impaired targeting of H-2K^{b} molecules to endolysosomes in the absence of TAP. These data suggested that a substantial fraction of MHC class I molecules interacted with CD74, facilitating their transport to the endolysosomal compartment of DCs, probably from the ER.

Demonstration of a direct molecular interaction between MHC class I and CD74 in DCs would further strengthen the argument that this is an as-yet-undescribed pathway of antigen presentation in DCs. To demonstrate this, BMDCs were obtained from various knockout and wild-type mice and labeled the cells with ³⁵S, then coimmunoprecipitated complexes bound to MHC class I (H-2K^{b}), MHC class II (I-A^{b}) or CD74 and identified the proteins in these complexes on the basis of their apparent molecular weight. Antibody to MHC class II immunoprecipitated the abundant 41-kilodalton (41-kDa) and 31-kDa isoforms of CD74 in Cd74^{+/+} DCs (FIG. 6c). Anti-H-2K^{b} also precipitated those same CD74 isoforms (FIG. 6c), which suggested that at any one time, CD74 was bound to a fraction of the total pool of MHC class I molecules in DCs. The two prominent proteins detected with a molecular size between 41 and 31 kDa may have been components of a MHC class I loading or transporting complex. Their sizes were consistent with those of H-2DM or H-2DO, that act as chaperones in MHC class II loading but their identities have not yet been conclusively determined. The 41- and 31-kDa forms of CD74 were not present in Cd74^{-/-} DCs (FIG. 6c), which indicated that they were indeed the reported isoforms of CD74 that have been shown to immunoprecipitate together with MHC class I and MHC class II molecules^{17-19,23}. In addition, the 41- and 31-kDa CD74 isoforms immunoprecipitated together with H-2K^{b} in Tap1^{-/-} DCs (FIG. 6c), which suggested that the binding of CD74 to MHC class I was not dependent on the peptide-transporter function of TAP. Finally, the CD74 isoforms precipitated together with MHC class I from β₂-microglobulin-deficient DCs (FIG. 6c), which suggested that CD74 was able to bind the folded β₂-microglobulin-associated MHC class I complex and the β₂-microglobulin-free MHC class I complex.

Immunoblot analysis was then used to confirm the identity of the CD74 isoforms bound to MHC class I molecules. Proteins were immunoprecipitated with anti-I-A^{b}, anti-H-2K^{b} and antibody to the region of the MHC class I molecule encoded by exon 8, as well as an irrelevant antibody to the transferrin receptor, followed by immunoblot analysis with anti-CD74 (FIG. 6d). As expected, CD74 associated with MHC class II (I-A^{b}) but not with the irrelevant protein transferrin receptor (FIG. 6d). CD74 was definitively identified as being associated with MHC class I (FIG. 6d), which confirmed that this interaction was detectable and stable under the conditions used in this immunoprecipitation procedure.

### A MHC Class I-CD74 Complex Forms in a Pre-Golgi Compartment

Next, to unequivocally demonstrate the kinetics and origin of the interaction between MHC class I and CD74, biochemical means was used to further deduce the intracellular compartment in which this interaction takes place. Proteins in the secretory pathway acquire resistance to endoglycosidase (Endo H) as they traffic from the ER through the Golgi compartment, and there they undergo cleavage by mannosidase II³⁵. It is well accepted that sensitivity to Endo H acts as an indication that proteins are localized to the ER or in ‘transitional elements’ between the ER and cis-Golgi. CD74-bound MHC class I from Cd74^{+/+} BMDCs was immunoprecipitated with a anti-CD74 or anti-MHC class I and treated the immunoprecipitates with Endo H, then did immunoblot analysis with anti-MHC class I or anti-CD74 to visualize the sensitivity of the MHC class I-CD74 complex to Endo H. We found that the MHC class I associated with CD74 was sensitive to Endo H (FIG. 6e,f). Furthermore, there was slightly more association of Endo H-resistant CD74 with MHC class I after treatment with chloroquine, as demonstrated by higher band intensities (FIG. 6f). Overall, these data suggested that the interaction of CD74 with MHC class I originated in the ER, where CD74 bound an ‘immature’ fraction of the MHC class I molecules and from there initiated trafficking to an endolysosomal compartment to mediate cross-presentation, T cell priming and primary immune responses^{8,13}.

CD74 does not Affect Internalization of MHC Class I

Finally, to determine the source of MHC class I that bound CD74, we investigated the role of CD74-mediated trafficking of MHC class I from the plasma membrane. To determine if CD74 functions in surface receptor recycling, the internalization of MHC class I in Cd74^{+/+} and Cd74^{-/-} DCs was monitored. BMDCs were stained with anti-H-2K^{b} and used flow cytometry to monitor internalization over time. Cd74^{+/+} and Cd74^{-/-} DCs had very similar dynamics of MHC class I internalization (FIG. 6g). This indicated that CD74 was not interacting with MHC class I at the cell surface to cause internalization into an intracellular compartment for cross-presentation. This complimented published studies that demonstrate a tyrosine-based motif in the cytoplasmic domain of MHC class I molecules is crucial for the internalization of recycling MHC class I molecules into the endolysosomal cross-priming compartment from the plasma membrane^{8,13} and thus demonstrated a unique and distinct pathway of CD74-dependent MHC class I trafficking.

### Discussion

The dichotomy of the presentation of exogenous peptides by MHC class II molecules versus the display of cytosolic peptides by MHC class I molecules has been revised^{6,8,36,37}. MHC class I cross-presentation not only demonstrated the blurring of this division but also shows that for specific cell types such as DCs, this phenomenon serves a major role in generating primary immune responses in vivo⁸. In addition, the presentation of endogenously derived peptides on MHC class II molecules demonstrates that MHC class I and class II pathways possibly intersect and that they may share the same antigen-loading compartments³⁸. Although CD74 is classically recognized as a major chaperone in presentation by MHC class II, CD74 and MHC class I have also been shown to interact^{17,18,39,40}. However, the physiological contribution of CD74 to MHC class I-mediated immune responses in vivo has not been investigated and the identification of a MHC class I-CD74 interaction was largely discounted as a biological curiosity. Here it has been demonstrated that CD74 contributed substantially to MHC class I cross-presentation pathways in DCs. These studies have identified a major role for CD74-dependent cross-priming in the generation of responses to viral and cell-associated antigens.

To assess CD4⁺ T cell independent CTL responses generated through DC cross-presentation, a model of infection with a low dose of VSV was used. Low viral doses mimic the physiological situation in which most DCs would presumably be spared from infection and other infected cells would act as antigenic peptide donors, which allows the delineation of direct or endogenous presentation versus cross-presentation. The observation that mice lacking CD74 were considerably impaired in their ability to generate MHC class I-restricted CTL responses, particularly to low viral doses at which cross-priming probably dominates over direct priming by DCs, supported the conclusion that MHC class I cross-presentation is the main mechanism by which antiviral CD8⁺ T cell-mediated immunity is generated under physiological conditions in vivo^{8,41}. We also confirmed the work of others and demonstrated that the responses of CTL to viruses such as VSV are CD4⁺ T cell independent^{26,27} and thus independent of the function of MHC class II-CD74 complexes.

The generation of bone marrow chimeras made it possible to study the activity of Cd74^{-/-} myeloid cell-derived DCs on a wild-type host background. Those studies led to the conclusion that the priming defect of CD74 was of DC origin and indicated that the deficiency was at the level of DC cross-presentation. Furthermore, CD74-dependent cross-priming was identified as an important MHC class I antigen-presentation pathway, as the absence of CD74 resulted in more than 50% fewer anti-VSV CTLs. In addition, the findings obtained by mouse chimeras supported the observation that CD74 deficiency impairs the generation of primary immune responses to VSV independently of the lower abundance of CD4⁺ T cells^{26,42}. This is in accordance with other published data demonstrating that in some cases, CD4⁺ T cells are required for secondary CTL population expansion but not primary population expansion⁴³. Costimulation of CD8⁺ CTLs by B7 molecules, along with stimulation of the T cell antigen receptor, can be sufficient to elicit CD8⁺ CTLs without T cell help²⁶. Alternatively, it is entirely possible that two distinct lineages of CD8⁺ CTL precursors exist whereby the CD4⁺ T cell-independent population provides the predominant response to various viruses, which results in no loss of CTL function in the absence of CD4⁺ T cells⁴².

It was found that the expression of a form of CD74 lacking its endosomal targeting signal failed to complement DC cross-presentation and priming of T cells. However, reconstitution with a wild-type CD74 molecule containing a functional endosomal targeting signal restored cross-priming, which supported the proposal of a mechanism whereby MHC class I was transported from the ER to the endolysosome by CD74. Additionally, the deficient activation of CD8⁺ T cells by Cd74^{-/-} DCs in Rag1^{-/-} mice that completely lack CD4⁺ T cells unequivocally demonstrated that the defect in DC cross-priming function was due to the absence of CD74. In our studies, CD74 did not seem to have a role in DC homing and motility in vivo but did mediate a physiologically important pathway for the CD74-dependent MHC class I cross-priming of CD8⁺ T cells by DCs.

Our studies have also provided evidence of an association between MHC class I molecules and CD74 in DCs under physiological conditions. They also suggested that after dissociation of the MHC class I-CD74 complex in endolysosomes, reassembly of the MHC class I heavy chain with β₂-microglobulin and antigenic peptides could then take place in the endolysosomal compartment⁴⁴. In this context, it was directly demonstrated that the MHC class I-CD74 complex remains assembled in vesicular-like compartments identified as late endosomes. Furthermore, it has been established that CD74 influences the presence of MHC class I in endolysosomes, which confirmed published observations that an MHC class I-CD74 interaction results in the targeting of a subset of MHC class I molecules to the endolysosomal pathway¹⁷.

The tyrosine internalization signal in the MHC class I cytoplasmic tail that has been previously described^{8,13,45} targets recycling MHC class I into the cross-priming compartment. In contrast to this mechanism, it is unlikely that a stable interaction between CD74 and MHC class I molecules occurs at the plasma membrane to direct recycling MHC class I, as the absence of CD74 in DCs did not seem to influence the internalization of MHC class I. Our results support a model whereby both the recycling of MHC class I from the plasma membrane, directed by a tyrosine internalization signal in the cytoplasmic domain, and the trafficking of MHC class I from the ER through binding to the CD74 chaperone contributes to the pool of peptide-receptive MHC class I in the endolysosomal pathway. Thus, in a manner analogous to that used by MHC class II molecules, the MHC class I-CD74 complex is formed in the ER and may be held in a conformation that masks peptide binding as it transits to the cross-priming compartment. Indeed, two independent studies have shown that CD74 peptides, including a smaller peptide derived from the core MHC class II associated CD74 peptide CLIP (MRMATPLLM), the portion of CD74 bound in the MHC class II binding groove, can be eluted from MHC class I molecules^{46,47}. Such peptides are therefore strong candidates for the MHC class I equivalents of CLIP. This CLIP-derived peptide may prevent premature peptide binding akin to MHC class II situation^{46,48}. In this model, after digestion and removal of CD74, MHC class I could be loaded with high-affinity cathepsin S-derived exogenous peptides¹¹ and progress to the cell surface, where they could efficiently prime CD8⁺ T cell precursors to become activated.

In summary, our results here and other published data^{8,49} emphasize the importance of the endolysosome as a principle compartment for cross-presentation in DCs, and our investigation here has formally established the structural and functional relevance of the MHC class I-CD74 interaction on the intracellular routing of MHC class I molecules and cross-priming function of DCs. Our observations have defined a previously unknown pathway for the priming of immune responses; future studies should completely elucidate this process. Our results are of considerable clinical relevance and suggest that targeting vaccine candidates to the endolysosomes of DCs would enhance priming for both MHC class I and MHC class II antigens and thereby improve the immunogenicity and efficacy of vaccines.

### Methods

Mice.

Cd74^{+/+} (H-2K^{b}) mice were from Charles River. The β₂-microglobulin-deficient B2m^{-/-}, Tap1^{-/-}, OT-I (H-2K^{b}) and Rag1^{-/-}(H-2K^{d}) mice were from Jackson Laboratory. Cd74^{-/-} (H-2K^{b}) mice were a gift from D. Mathis. For chimeric mice, donor bone marrow was depleted of mature T cells with anti-Thy-1 (MRC OX-7; Abcam) and injected (1×10⁷ cells) into sublethally irradiated recipients (1,200 rads). Peripheral T cell subsets were analyzed by flow cytometry after being stained with anti-CD8 (53-6.7; BD Pharmingen) and anti-CD4 (GK1.5, BD Pharmingen). For depletion of CD4⁺ cells, before immunization and 48 h before T cell assessment, mice were injected with 100 μg anti-CD4 (GK1.5)⁵⁰. All studies followed guidelines set by the University of British Columbia's Animal Care Committee and the Canadian Council on Animal Care.

Virol Infection.

VSV was injected intraperitoneally (at 1×10⁵ to 2×10⁵ of a dose that infects 50% of a tissue culture cell monolayer). At 6 d after infection, splenocytes were stained with anti-CD8 (53-6.7) and H-2K^{b}-VSVNP(52-59) or H-2K^{b}-OVA(257-264) iTAg tetramer (immunomics-BeckmanCoulter) and analyzed with a FACSCalibur (Becton Dickinson) and FlowJo software. Splenocytes were further cultured for 5 d with 1 μM OVA(257-264) (SIINFEKL) or VSVNP(52-59) (RGYVYQGL), followed by tetramer staining as described above. Cytotoxicity assays were done as described⁸.

Uptake Assay.

BMDCs were generated as described⁸. Cells were incubated for 30 min at 4° C. or at 37° C. with OVA-Alexa Fluor 488 (30 μg/ml; Invitrogen). OVA uptake was analyzed by flow cytometry.

Cross-Presentation Assay.

BMDCs were generated as described⁸ or splenic DCs were isolated with CD11c⁺ magnetic beads (Miltenyi Biotech). DCs were incubated for 15 h with OVA (Worthington) and, where indicated, with 100 μM chloroquine. DCs were stained with Fc Block (PharMingen), then with anti-H-2K^{b} (AF.6-88.5), anti-CD80 (16-10A1), anti-CD86 (GL1), anti-CD40 (3/23) all from BD Pharmingen or anti-H-2K^{b}-OVA(257-264) (25.D1.16; a gift from J. Yewdell) and analyzed by flow cytometry. For cross-priming assays, DCs were incubated with OVA, GM-CSF (granulocyte-macrophage colony-stimulating factor; 15 ng/ml; Sigma) and TNF (10 ng/ml) or interferon-γ (R&D Systems). Activation of B3Z T cells (a gift from N. Shastri) was assessed as described⁸.

For in vivo studies, Cd74^{+/+} and Cd74^{-/-} BMDCs were incubated for 2 h with OVA or OVA(257-264) (10 mg/ml each) and were injected intravenously into Rag1^{-/-} BALB/c mice (1×10⁷ cells). After 24 h, OT-I T cells were labeled with 2.5 μM CFSE (carboxyfluorescein diacetate succinimidyl ester; Molecular Probes) and were injected intravenously into mice (5×10⁶ cells). The proliferation of OT-I T cells in the spleen was assessed by flow cytometry 3 d later as CFSE dilution. For confirmation of localization to spleen, CFSE-labeled DCs were injected intravenously into Rag1^{-/-} BALB/c mice. After 2 h, the presence of CFSE⁺ cells in the spleen was assessed with flow cytometry.

Confocal Microscopy.

Spleen-derived DCs were isolated, fixed and made permeable as described⁸. For analysis of cross-presentation, DCs were incubated with for 10 h with OVA (5 mg/ml) with or without TNF (10 ng/ml). Where needed, DCs were treated with 50 μM chloroquine for 72 h before processing³⁴. Cells were stained with anti-H-2K^{b} (AF.6-88.5), anti-CD74 (ln-1; Fitzgerald), anti-LAMP-1 (N19; Santa Cruz Biotechnology) or anti-H-2K^{b}-OVA(257-264) (25.D1.16). Alexa Fluor 488- or Alexa Fluor 568-conjugated rabbit anti-mouse (A-11029, A-11031; Molecular Probes), Alexa Fluor 488- or Alexa Fluor 568-conjugated rabbit anti-goat (A-11078, A-11079; Molecular Probes) or Alexa Fluor 488-conjugated goat anti-mouse (A-11001; Molecular Probes) were used as secondary antibodies. Images were acquired with a Nikon-C1, TE2000-U ICM and EZ-C1 software. Data were analyzed with ImageJ.1, Open/ab and Adobe Photoshop. The fluorescence intensity of individual colors is presented as a percent of total fluorescence intensity.

Proliferation Assay.

BMDCs derived from C3H/He mice (H-2K^{k}) were incubated for 15 h with OVA (10 mg/ml) and were injected intraperitoneally into mice (5×10⁶ cells). OT-I T cells were labeled and injected intravenously as described above. The proliferation of OT-I T cells was assessed 3 d later by flow cytometry as CFSE dilution.

Transfection.

Immature BMDCs were transfected with pBabe vector (a gift from I. Shachar) containing full-length mouse CD74 (p31 isoform) or CD74 lacking amino acids 2-17 through use of an Amaxa Mouse Dendritic Cell Nucleofector kit. At 1 d after electroporation, DCs were incubated for 8 h with OVA (20 mg/ml) or OVA(257-264) (1 μM), then were incubated for 3 d with CFSE-labeled OT-I-CD8⁺ T cells. CFSE dilution was assessed by flow cytometry.

Immunoprecipitation.

BMDCs were incubated for 1 h methionine- and cysteine-free media, then were pulsed with for 30 min with [³⁵S]methionine (300 μCi/ml), then lysed in 0.5% (vol/vol) Nonidet P-40 in buffer (120 mM NaCl, 4 mM MgCl₂ and 20 mM Tris-HCl, pH 7.6) containing a protease inhibitor ‘cocktail’ (Roche) and PMSF (phenylmethyl sulfonyl fluoride; 40 μg/ml). Where indicated, DCs were incubated with 100 μM chloroquine overnight before lysis. Cell lysates were precleared by incubation overnight with normal rabbit serum and protein ASepharose (Pharmacia). Anti-H-2K^{b} recognizing fully folded MHC class I (AF6.88.5; BD Pharmingen), antibody to sequence encoded by exon 8 that recognizes all MHC class I (from D. Williams and B. Barber), anti-I-A-I-E (M5/114.15.2; Becton Dickinson), anti-CD74 (ln-1; Fitzgerald) and antibody to transferrin receptor (H68.4; Invitrogen) were used for immunoprecipitation. Samples were separated by 10-12% SDS-PAGE. Gels were fixed, enhanced with Amplify (Amersham Biosciences), dried and exposed to Kodak XMR autoradiographic film. Alternatively, samples were transferred to a nitrocellulose membrane and analyzed by immunoblot with anti-CD74 (ln-1; Fitzgerald) or anti-MHC class I (KH95; SantaCruz Biotechnology). Samples were digested endoglycosidase H_{f} according to the manufacturer's protocol (New England Biolabs). Whole-cell lysates were analyzed by immunoblot as a positive control. Donkey antibody to mouse immunoglobulin G (926-32212; Li-Cor Biosciences) and goat antibody to mouse rat immunoglobulin G (A21096; Invitrogen) were used as secondary antibodies. Blots were visualized with the Odyssey Infrared Imaging.

MHC Class I Internalization.

BMDCs were stained with Fc Block (BD Pharmingen), then were labeled for 30 min at 0° C. with biotinylated anti-H-2K^{b} (AF6-88.5; BD Pharmingen). Samples were incubated at 37° C. or 0° C. At the appropriate time, DCs were fixed in 2% (vol/vol) paraformaldehyde and labeled with streptavidin-phycoerythrin, then were examined by flow cytometry. Data were analyzed with Flowio software to calculate the amount of internalized MHC class I.

Statistical Analysis.

Student's t-test was used to compare the difference between populations. Differences were considered statistically significant when the P value was less than 0.05 (two-tailed).

### REFERENCES

1. Guagliardi, L. E. et al. Co-localization of molecules involved in antigen processing and presentation in an early endocytic compartment. Nature 343, 133-139 (1990).
2. Kovacsovics-Bankowski, M. & Rock, K. L. A phagosome-to-cytosol pathway for exogenous antigens presented on MHC class I molecules. Science 267, 243-246 (1995).
3. Ackerman, A. L., Kyritsis, C., Tampe, R. & Cresswell, P. Early phagosomes in dendritic cells form a cellular compartment sufficient for cross presentation of exogenous antigens. Proc. Natl. Acad. Sci. USA 100, 12889-12894 (2003).
4. Guermonprez, P. et al. ER-phagosome fusion defines an MHC class I cross-presentation compartment in dendritic cells. Nature 425, 397-402 (2003).
5. Houde, M. et al. Phagosomes are competent organelles for antigen cross-presentation. Nature 425, 402-406 (2003).
6. Pfeifer, J. D. et al. Phagocytic processing of bacterial antigens for class I MHC presentation to T cells. Nature 361, 359-362 (1993).
7. Song, R. & Harding, C. V. Roles of proteasomes, transporter for antigen presentation (TAP), and β2-microglobulin in the processing of bacterial or particulate antigens via an alternate class I MHC processing pathway. J. Immunol. 156, 4182-4190 (1996).
8. Lizée, G. et al. Control of dendritic cell cross-presentation by the major histocompatibility complex class I cytoplasmic domain. Nat. Immunol. 4, 1065-1073 (2003).
9. Gagnon, E. et al. Endoplasmic reticulum-mediated phagocytosis is a mechanism of entry into macrophages. Cell 110, 119-131 (2002).
10. Touret, N. et al. Quantitative and dynamic assessment of the contribution of the ER to phagosome formation. Cell 123, 157-170 (2005).
11. Shen, L., Sigal, L. J., Boes, M. & Rock, K. L. Important role of cathepsin S in generating peptides for TAP-independent MHC class I crosspresentation in vivo. Immunity 21, 155-165 (2004).
12. Cebrian, I. et al. Sec22b regulates phagosomal maturation and antigen crosspresentation by dendritic cells. Cell 147, 1355-1368 (2011).
13. Basha, G. et al. MHC class I endosomal and lysosomal trafficking coincides with exogenous antigen loading in dendritic cells. PLoS ONE 3, e3247 (2008).
14. Chiu, I., Davis, D. M. & Strominger, J. L. Trafficking of spontaneously endocytosed MHC proteins. Proc. Natl. Acad. Sci. USA 96, 13944-13949 (1999).
15. Reid, P. A. & Watts, C. Cycling of cell-surface MHC glycoproteins through primaquine-sensitive intracellular compartments. Nature 346, 655-657 (1990).
16. Bakke, O. & Dobberstein, B. MHC class II-associated invariant chain contains a sorting signal for endosomal compartments. Cell 63, 707-716 (1990).
17. Sugita, M. & Brenner, M. B. Association of the invariant chain with major histocompatibility complex class I molecules directs trafficking to endocytic compartments. J. Biol. Chem. 270, 1443-1448 (1995).
18. Vigna, J. L., Smith, K. D. & Lutz, C. T. Invariant chain association with MHC class I: preference for HLA class I/β2-microglobulin heterodimers, specificity, and influence of the MHC peptide-binding groove. J. Immunol. 157, 4503-4510 (1996).
19. Kleijmeer, M. J. et al. Antigen loading of MHC class I molecules in the endocytic tract. Traffic 2, 124-137 (2001).
20. Zwickey, H. L. & Potter, T. A. Antigen secreted from noncytosolic Listeria monocytogenes is processed by the classical MHC class I processing pathway. J. Immunol. 162, 6341-6350 (1999).
21. MacAry, P. A. et al. Mobilization of MHC class I molecules from late endosomes to the cell surface following activation of CD34-derived human Langerhans cells. Proc. Natl. Acad. Sci. USA 98, 3982-3987 (2001).
22. Tourne, S. et al. Biosynthesis of major histocompatibility complex molecules and generation of T cells in Ii TAP1 double-mutant mice. Proc. Natl. Acad. Sci. USA 93, 1464-1469 (1996).
23. Reber, A. J., Turnquist, H. R., Thomas, H. J., Lutz, C. T. & Solheim, J. C. Expression of invariant chain can cause an allele-dependent increase in the surface expression of MHC class I molecules. Immunogenetics 54, 74-81 (2002).
24. Vitalis, T. Z. et al. Using the TAP component of the antigen-processing machinery as a molecular adjuvant. PLoS Pathog. 1, e36 (2005).
25. van Kaer, L., Ashton-Rickardt, P. G., Ploegh, H. L. & Tonegawa, S. TAP1 mutant mice are deficient in antigen presentation, surface class I molecules, and CD4⁻8⁺ T cells. Cell 71, 1205-1214 (1992).
26. McAdam, A. J., Farkash, E. A., Gewurz, B. E. & Sharpe, A. H. B7 costimulation is critical for antibody class switching and CD8⁺ cytotoxic T-lymphocyte generation in the host response to vesicular stomatitis virus. J. Virol. 74, 203-208 (2000).
27. Marzo, A. L. et al. Fully functional memory CD8 T cells in the absence of CD4 T cells. J. Immunol. 173, 969-975 (2004).
28. Faure-Andre, G. et al. Regulation of dendritic cell migration by CD74, the MHC class II-associated invariant chain. Science 322, 1705-1710 (2008).
29. Benvenuti, F. et al. Requirement of Rac1 and Rac2 expression by mature dendritic cells for T cell priming. Science 305, 1150-1153 (2004).
30. Shastri, N. & Gonzalez, F. Endogenous generation and presentation of the ovalbumin peptide/Kb complex to T cells. J. Immunol. 150, 2724-2736 (1993).
31. Sallusto, F., Cella, M., Danieli, C. & Lanzavecchia, A. Dendritic cells use macropinocytosis and the mannose receptor to concentrate macromolecules in the major histocompatibility complex class II compartment: downregulation by cytokines and bacterial products. J. Exp. Med. 182, 389-400 (1995).
32. Brossart, P. & Bevan, M. J. Presentation of exogenous protein antigens on major histocompatibility complex class I molecules by dendritic cells: pathway of presentation and regulation by cytokines. Blood 90, 1594-1599 (1997).
33. Merzougui, N., Kratzer, R., Saveanu, L. & van Endert, P. A proteasome-dependent, TAP-independent pathway for cross-presentation of phagocytosed antigen. EMBO Rep. 12, 1257-64 doi:10.1038/embor.2011.203 (2011).
34. Loss, G. E. Jr. & Sant, A. J. Invariant chain retains MHC class II molecules in the endocytic pathway. J. Immunol. 150, 3187-3197 (1993).
35. Kornfeld, R. & Kornfeld, S. Assembly of asparagine-linked oligosaccharides. Annu. Rev. Biochem. 54, 631-664 10.1146/annurev.bi.54.070185.003215 (1985).
36. Rock, K. L., Gamble, S. & Rothstein, L. Presentation of exogenous antigen with class I major histocompatibility complex molecules. Science 249, 918-921 (1990).
37. van Lith, M., van Ham, M. & Neefjes, J. Stable expression of MHC class I heavy chain/HLA-DO complexes at the plasma membrane. Eur. J. Immunol. 33, 1145-1151 (2003).
38. Nuchtern, J. G., Biddison, W. E. & Klausner, R. D. Class II MHC molecules can use the endogenous pathway of antigen presentation. Nature 343, 74-76 10.1038/343074a0 (1990).
39. Cerundolo, V., Elliott, T., Elvin, J., Bastin, J. & Townsend, A. Association of the human invariant chain with H-2 Db class I molecules. Eur. J. Immunol. 22, 2243-2248 (1992).
40. Powis, S. J. CLIP-region mediated interaction of Invariant chain with MHC class I molecules. FEBS Lett 580, 3112-3116 (2006).
41. Sigal, L. J. & Rock, K. L. Bone marrow-derived antigen-presenting cells are required for the generation of cytotoxic T lymphocyte responses to viruses and use transporter associated with antigen presentation (TAP)-dependent and -independent pathways of antigen presentation. J. Exp. Med. 192, 1143-1150 (2000).
42. Buller, R. M., Holmes, K. L., Hugin, A., Frederickson, T. N. & Morse, H. C., III. Induction of cytotoxic T-cell responses in vivo in the absence of CD4 helper cells. Nature 328, 77-79 10.1038/328077a0 (1987).
43. Janssen, E. M. et al. CD4⁺ T cells are required for secondary expansion and memory in CD8⁺ T lymphocytes. Nature 421, 852-856 (2003).
44. Machold, R. P. & Ploegh, H. L. Intermediates in the assembly and degradation of class I major histocompatibility complex (MHC) molecules probed with free heavy chain-specific monoclonal antibodies. J. Exp. Med. 184, 2251-2259 (1996).
45. Reinicke, A. T., Omilusik, K. D., Basha, G. & Jefferies, W. A. Dendritic cell cross-priming is essential for immune responses to Listeria monocytogenes. PLoS ONE 4, e7210 10.1371/journal.pone.0007210 (2009).
46. Luckey, C. J. et al. Differences in the expression of human class I MHC alleles and their associated peptides in the presence of proteasome inhibitors. J. Immunol. 167, 1212-1221 (2001).
47. Krüger, T. et al. Lessons to be learned from primary renal cell carcinomas: novel tumor antigens and HLA ligands for immunotherapy. Cancer Immunol. Immunother. 54, 826-836 (2005).
48. Busch, R., Cloutier, I., Sekaly, R. P. & Hammerling, G. J. Invariant chain protects class II histocompatibility antigens from binding intact polypeptides in the endoplasmic reticulum. EMBO J. 15, 418-428 (1996).
49. Savina, A. et al. NOX2 controls phagosomal pH to regulate antigen processing during crosspresentation by dendritic cells. Cell 126, 205-218 (2006).

50. Rashid, A., Auchincloss, H. Jr. & Sharon, J. Comparison of GK1.5 and chimeric rat/mouse GK1.5 anti-CD4 antibodies for prolongation of skin allograft survival and suppression of alloantibody production in mice. J. Immunol. 148, 1382-1388 (1992).

1-3. (canceled)

4. A fusion protein comprising an antigen or fragment thereof and a CD74 endolysosomal targeting sequence.

5-7. (canceled)

8. The fusion protein of claim 4, where the CD74 is human CD74.

9. The fusion protein of claim 4, where the CD74 is murine CD74.

10. The fusion protein of claim 4, where the antigen is viral antigen.

11. A pharmaceutical composition, comprising:
a) a fusion protein comprising an antigen or fragment thereof and a CD74 endolysosomal targeting sequence;
b) an expression vector which expresses CD74; and/or
c) dendritic cells which have been genetically engineered to over-express CD74,
and a pharmaceutically acceptable diluent or carrier.

12. The pharmaceutical composition of claim 11, where the dendritic cells comprise an expression vector which expresses CD74.

13. The pharmaceutical composition of claim 11, where the CD74 is human CD74.

14. The pharmaceutical composition of claim 11, where the CD74 is murine CD74.

15. The pharmaceutical composition of claim 11, where the antigen is viral antigen.

16. A method of treating a disease caused by an intracellular pathogen, comprising administering to an individual a pharmaceutical composition of claim 11.

17. The method of claim 16, where the disease caused by the intracellular pathogen is a viral infection.

18. The method of claim 16, where the disease caused by the intracellular pathogen is HIV infection, mycobacterial infection, plasmodium infection, or malaria.
